(19) United States
(12) Patent Application Publication
(10) Pub. No.: US 2006/0211971 A1
(43) Pub. Date: Sep. 21, 2006

(54) Pillow for the delivery of blood clotting materials to a wound site

(75) Inventors: Jeffrey L. Horn, Rocky Hill, CT (US); Raymond J. Huey, Orange, CT (US)

Correspondence Address: MICHAUD-DUFFY GROUP LLP, 306 INDUSTRIAL PARK ROAD, SUITE 206, MIDDLETOWN, CT, 06457, US

(73) Assignee: Z-Medica, LLC, Wallingford, CT (US)

(21) Appl. No.: 11/082,716

(22) Filed: Mar. 16, 2005

(51) Int. Cl. A61F 15/00; A61F 13/00

(52) U.S. Cl. 602/41

(57) ABSTRACT

A pillow for supporting a wounded victim at a wound site incorporates a molecular sieve material which is at least partially exposed so that blood flowing from a wounded area supported by the pillow contacts the molecular sieve material to facilitate the clotting of blood flowing from the wound. A removable flexible casing for a pillow incorporates a molecular sieve material capable of providing a clotting function to the blood. An overlay comprising a molecular sieve material can be placed on top of a conventional pillow, on a gurney, or on any other surface. A method of treating a bleeding wound includes providing a pillow having molecular sieve material in particle form retained therein and using the pillow to support a bleeding wound such that blood flowing from the wound comes into contact with the molecular sieve material, thereby causing the blood to coagulate.

### TECHNICAL FIELD

The present invention relates generally to blood clotting devices and, more particularly, to a pillow-like apparatus that allows blood clotting materials incorporated into the pillow-like device to be brought into contact with a wound site, particularly a head wound. The present invention is also directed to methods for the use of such pillows as bleeding control devices.

### BACKGROUND OF THE INVENTION

Blood is a liquid tissue that includes red cells, white cells, corpuscles, and platelets dispersed in a liquid phase. The liquid phase is plasma, which includes acids, lipids, solublized electrolytes, and proteins. The proteins are suspended in the liquid phase and can be separated out of the liquid phase by any of a variety of methods such as filtration, centrifugation, electrophoresis, and immunochemical techniques. One particular protein suspended in the liquid phase is fibrinogen. When bleeding occurs, the fibrinogen reacts with water and thrombin (an enzyme) to form fibrin, which is insoluble in blood and polymerizes to form clots.

In a wide variety of circumstances humans (as well as animals) can be wounded. Often bleeding is associated with such wounds. In some circumstances, the wound and the bleeding are minor, and normal blood clotting functions in addition to the application of simple first aid are all that is required. Unfortunately, however, in other circumstances substantial bleeding can occur. These situations usually require specialized equipment and materials as well as personnel trained to administer appropriate aid. If such aid is not readily available, excessive blood loss can occur. When bleeding is severe, sometimes the immediate availability of equipment and trained personnel is still insufficient to stanch the flow of blood in a timely manner.

In an effort to address the above-described problems, materials and devices have been developed for controlling excessive bleeding in situations where conventional aid is unavailable or less than optimally effective. Although these materials and devices have been shown to be somewhat successful, they are sometimes not effective enough for traumatic wounds and tend to be expensive. Furthermore, these materials are sometimes ineffective in some situations and can be difficult to apply as well as remove from a wound.

In situations in which traumatic wounds are experienced (such as automobile or motorcycle accidents) where multiple wounds are inflicted on a victim over multiple portions of the victim's body, some wounds may not be immediately noticeable. First aid may be administered to treat the visible wounds of the victim, but other wounds may be overlooked. In particular, a victim's wounds to his torso, legs, and face may be attended to before and during transport of the victim to a medical facility. During the transport or after the victim is moved, other less visible wounds, e.g., wounds on the back of the head or neck, may become apparent. Particularly with regard to lacerations on the back of the scalp that are hidden by the victim's hair, blood loss may be unnecessarily substantial before the laceration is discovered.

Based on the foregoing, it is a general object of the present invention to provide devices for controlling bleeding and methods of their use that overcome or improve upon the prior art.

### SUMMARY OF THE INVENTION

According to one aspect, the present invention resides in a pillow for supporting a wounded victim at a wound site. As used herein, the word “pillow” should be broadly construed to encompass pillows, pillow cases, overlays to be positioned on a pillow, or any other device upon which a person's (particularly an injured person's) head or other body part may rest. The pillow incorporates a molecular sieve material which is at least partially exposed so that blood flowing from a wounded area supported by the pillow contacts the molecular sieve material which facilitates the clotting of blood flowing from the wound. The molecular sieve material is retained in a mesh structure on a surface of the pillow on which the injured person's head or other body part rests.

Other aspects of the present invention include a removable flexible casing for a pillow. The flexible casing incorporates a molecular sieve material capable of providing a clotting function to the blood. The flexible casing can be maintained in sterile packaging until it is needed, at which time it can be removed from the packaging and used to encase a pillow. As above, the pillow encased in the flexible casing is used to support a wounded victim at the wound site, and the molecular sieve material is retained in a mesh structure on a surface of the flexible casing on which the injured victim's head or other body part rests.

In still another embodiment of the present invention, an overlay can be provided. The overlay can be placed on top of a conventional pillow, on a gurney, or on any other surface. The overlay has a first outermost surface comprised of a mesh material and a generally opposed second, impermeable outer surface. A hemostatic agent, preferably in the form of a molecular sieve material, is retainably located between the first and second outer surfaces. Accordingly, when the mesh first outer surface is placed in contact with a bleeding wound, the blood will contact the molecular sieve material thereby causing the blood coagulate. The overlay could also incorporate an absorbent layer between the second outer surface and the molecular sieve material. Moreover, fastening means such as hook-and-loop material, adhesives, or elastic bands or straps can also be incorporated to secure the overlay in position. The above-described pillows also can be of a length sufficient to wrap around a wound area, such as for example, a victim's head.

In yet another aspect of the present invention, a method of treating a bleeding wound includes providing a pillow having molecular sieve material in particle form retained therein and using the pillow to support a bleeding wound such that blood flowing from the wound comes into contact with the molecular sieve material, thereby causing the blood to coagulate.

Surprisingly, bleeding wounds to the back of a victim's head are often missed by emergency services personnel when treating a victim with other injuries who is perhaps covered with blood or other debris from, for example, an automobile accident. An advantage of the present invention is that a wound to the back of a victim's head would be treated to stop bleeding merely by placing the victim's head on the pillow of the present invention. By laying the victim such that the wound engages the zeolite (or other molecular sieve) material in the pillow clotting of blood emanating from the wound is facilitated regardless of whether or not the personnel treating the wound are aware of its existence.

Another advantage of the present invention is that the proper dose of molecular sieve material can be readily applied to an open wound. Particularly when the device is a pre-packaged pillow containing zeolite material, the device can be readily removed from sterilized packaging and used to support the victim directly at the points from which blood emanates to facilitate clotting of the blood without spilling powder or pellets outside the wound area. Guesswork, estimation, or calculation of the amounts of molecular sieve material for application to a bleeding wound is eliminated. Accordingly, little or no molecular sieve material is wasted.

### BRIEF DESCRIPTION OF THE DRAWINGS

FIG. 1 is a perspective view of a blood clotting pillow of the present invention.

FIG. 2 is a side sectional view of the pillow of FIG. 1.

FIG. 3 is a schematic representation of a permeable layer of the pillow of FIG. 1.

FIG. 4 is a side view of a pillow incorporating molecular sieve particles retained in permeable layers attached to both sides of the pillow.

FIG. 5 is a perspective view of a pillow case incorporating the molecular sieve particles, the pillow case being sized to receive a pillow.

FIG. 6 is an exploded perspective view of another embodiment of the present invention.

FIG. 7 is an exploded perspective view of the embodiment of FIG. 6 showing resilient or elastic straps attached to the pillow.

### DETAILED DESCRIPTION OF THE PREFERRED EMBODIMENTS

Disclosed herein are devices and methods for delivering materials to wounds to promote the clotting of blood and the dressing of the wounds. The devices generally comprise pillows that can be used to support victims at bleeding wound sites such that materials incorporated into the pillows are contacted by the tissue of the wound to minimize or stop blood flow by absorbing at least portions of the liquid phases of the blood, thereby promoting clotting. One pillow is a cushioning support device that comprises a resilient core that may be defined by open cell or closed cell foam material. At least a portion of the outer surface of the resilient core is covered by a permeable layer having a particulate molecular sieve material retained therein such that at least a portion of the particulate molecular sieve material can be maintained in direct contact with blood emanating from a wound through the permeable layer.

The molecular sieve material used in the present invention may be a synthetic polymer gel, cellulosic material, porous silica gel, porous glass, alumina, hydroxyapatite, calcium silicate, zirconia, zeolite, or the like. Exemplary synthetic polymers include, but are not limited to, stylene-divinylbenzene copolymer, cross-linked polyvinyl alcohol, cross-linked polyacrylate, cross-linked vinyl ether-maleic anhydride copolymer, cross-linked stylene-maleic anhydride copolymer or cross-linked polyamide, and combinations thereof.

The molecular sieve material is preferably a zeolite. Other molecular sieve materials that may be used include, but are not limited to, faujasite. As used herein, the term “zeolite” refers to a crystalline form of aluminosilicate having the ability to be dehydrated without experiencing significant changes in the crystalline structure. The zeolite may include one or more ionic species such as, for example, calcium and sodium moieties. Typically, the zeolite is a friable material that is about 90% by weight calcium and about 10% by weight sodium. The calcium portion contains crystals that are about 5 angstroms in size, and the sodium portion contains crystals that are about 4 angstroms in size. The preferred molecular structure of the zeolite is an “A-type” crystal, namely, one having a cubic crystalline structure that defines round or substantially round openings.

The zeolite may be mixed with or otherwise used in conjunction with other materials having the ability to be dehydrated without significant changes in crystalline structure. Such materials include, but are not limited to, magnesium sulfate, sodium metaphosphate, calcium chloride, dextrin, a polysaccharide, combinations of the foregoing materials, and hydrates of the foregoing materials.

Zeolites for use in the disclosed applications may be naturally occurring or synthetically produced. Numerous varieties of naturally occurring zeolites are found as deposits in sedimentary environments as well as in other places. Naturally occurring zeolites that may be applicable to the compositions described herein include, but are not limited to, analcite, chabazite, heulandite, natrolite, stilbite, and thomosonite. Synthetically produced zeolites that may also find use in the compositions and methods described herein are generally produced by processes in which rare earth oxides are substituted by silicates, alumina, or alumina in combination with alkali or alkaline earth metal oxides.

Various materials may be mixed with, associated with, or incorporated into the zeolites to maintain an antiseptic environment at the wound site or to provide functions that are supplemental to the clotting functions of the zeolites. Exemplary materials that can be used include, but are not limited to, pharmaceutically-active compositions such as antibiotics, antifungal agents, antimicrobial agents, anti-inflammatory agents, analgesics (e.g., cimetidine, chloropheniramine maleate, diphenhydramine hydrochloride, and promethazine hydrochloride), bacteriostatics, compounds containing silver ions, and the like. Other materials that can be incorporated to provide additional hemostatic functions include ascorbic acid, tranexamic acid, rutin, and thrombin. Botanical agents having desirable effects on the wound site may also be added.

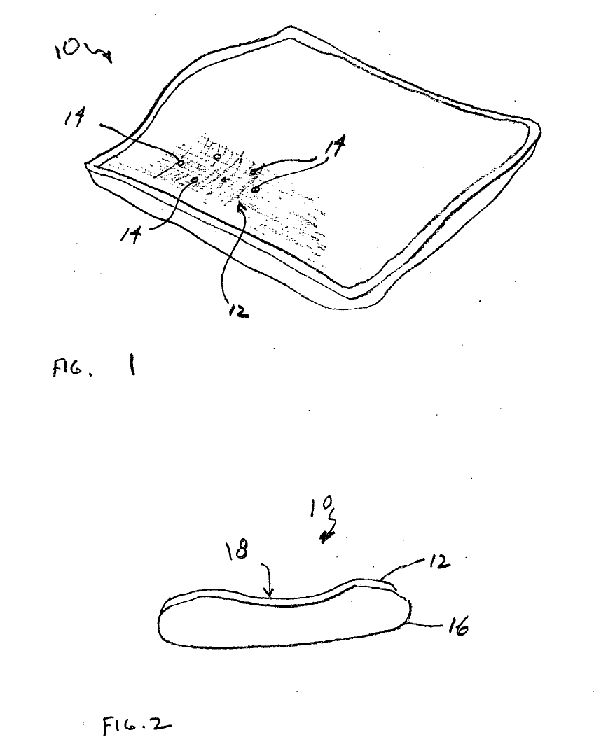

In one embodiment of the present invention shown in FIG. 1, a pillow for supporting a wounded victim at the wound site to facilitate the clotting of blood directly at the wound site is shown at reference numeral 10. The pillow 10 is a cushioning support having a permeable layer 12. When used to support a victim at the site of a bleeding wound, the permeable layer 12 allows blood to pass through the permeable layer and contact blood clotting zeolite (or other molecular sieve) material retained therein. The permeable layer 12 includes openings that are capable of retaining the zeolite particles 14 therein while allowing blood to flow through. As illustrated, the permeable layer 12 is shown as a mesh material, and only a few zeolite particles 14 are shown. Sealed packaging (not shown) provides a sterile environment for storing the pillow 10 until it can be used.

The permeable layer 12 is defined by interconnected strands, filaments, or strips of material. The strands, filaments, or strips can be interconnected in any one or a combination of manners including, but not limited to, being woven into a gauze, intertwined, integrally-formed, and the like. Preferably, the interconnection is such that the permeable layer 12 can flex to conform to and retain the shape of the wound while substantially maintaining the dimensions of the openings defined thereby. The material from which the strands, filaments or strips are fabricated may be a polymer (e.g., nylon, polyethylene, polypropylene, polyester, or the like), metal, fiberglass, or an organic substance (e.g., cotton, wool, silk, or the like).

The zeolite particles 14 are substantially spherical or irregular in shape (e.g., balls, beads, pellets, or the like) and about 0.2 millimeters (mm) to about 10 mm in diameter, preferably about 1 mm to about 7 mm in diameter, and more preferably about 2 mm to about 5 mm in diameter. In any embodiment (balls, beads, pellets, etc.), less particle surface area is available to be contacted by blood as the particle size is increased. Therefore, the rate of clotting can be controlled by varying the particle size. Furthermore, the adsorption of moisture (which also has an effect on the exothermic effects of the zeolite) can also be controlled.

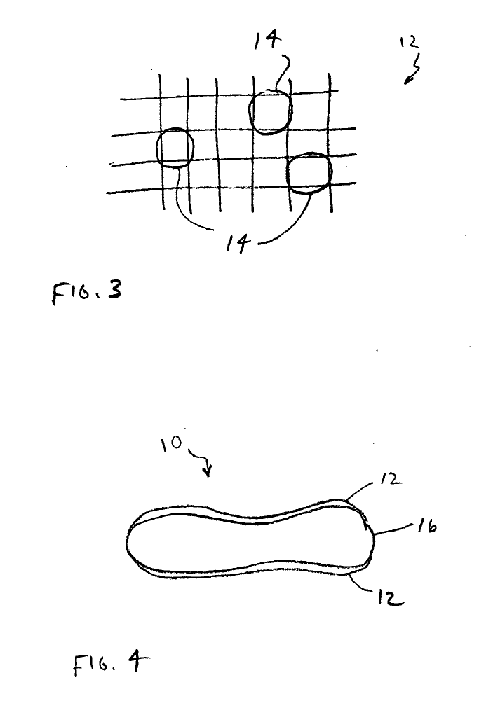

Referring to FIG. 2, the permeable layer 12 of the pillow 10 encases a resilient core 16. The resilient core 16 is a mass of resilient material sized and dimensioned such that at least a portion of the victim can be rested thereon. More specifically, the resilient core 16 is of sufficient height, width, and depth to comfortably accommodate the victim's head when the victim is in a supine, prone, or reclined position or when the victim is laying on his side. At least one major face 18 of the resilient core 16 may be concave or similarly contoured to cradle the head or support the neck of the victim. The resilient core 16 may comprise open-cell or closed-cell foam. Typical foam materials include, but are not limited to, polyurethanes, polyethylenes, sponge rubbers, combinations of the foregoing materials, and the like.

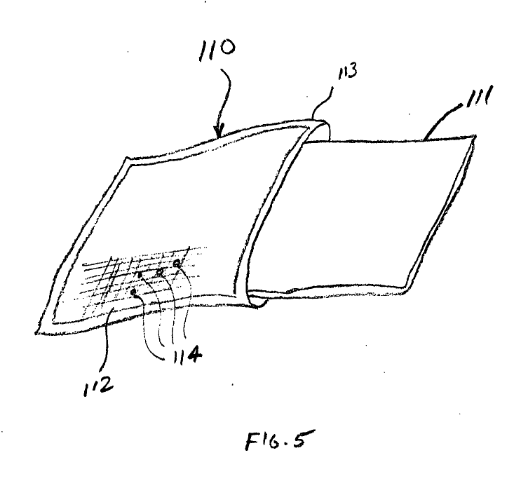

Referring now to FIG. 3, the openings defined by the permeable layer 12 are dimensioned to retain the zeolite particles 14 but to accommodate the flow of blood therethrough. Because the zeolite particles 14 may be tightly packed into the permeable layer 12, the particles may partially extend through the openings. However, it is not a requirement of the present invention that the zeolite particles 14 protrude through the openings of the permeable layer 12. If the zeolite particles 14 do extend through the openings, the particles are able to directly contact tissue to which the pillow is applied. Thus, blood emanating from the tissue immediately contacts the zeolite particles 14, and the water phase thereof is wicked into the zeolite material, thereby facilitating the clotting of the blood.

Referring now to FIG. 4, the pillow 10 may include permeable layers 12 attached to both sides of the resilient core 16. As above, one or both of the major faces 18 may be concave or contoured.

Referring now to FIGS. 1-4, to utilize the pillow 10 to support a victim at a bleeding wound, the pillow is removed from the packaging (if any) and placed on a support surface (e.g., the ground, a gurney, or the like) such that the victim can be laid or otherwise supported by the pillow. The zeolite particles 14 in the permeable layer 12 contact the tissue of the wound and/or the blood, and at least a portion of the liquid phase of the blood is adsorbed by the zeolite material, thereby promoting the clotting of the blood.

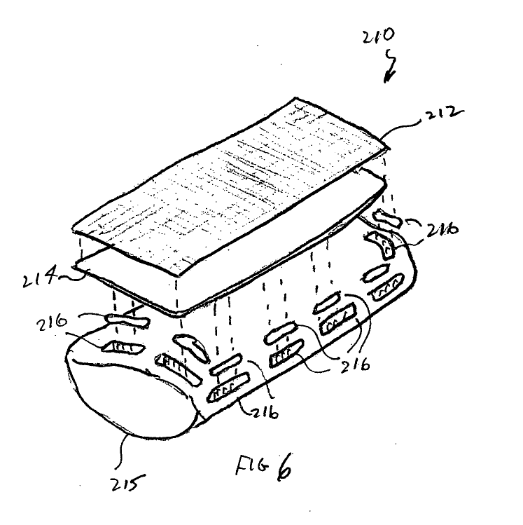

Referring now to FIG. 5, a pillowcase having zeolite material incorporated therein is shown at 110. The pillowcase 110, which can be fitted over a pillow 111, includes a flexible casing 113 having a permeable layer 112 attached thereto. The flexible casing 113 may be in the form of a bag having an opening through which the pillow 111 can be inserted. The flexible casing 113 may be made closable using a zipper, hook-and-loop material, snaps, or a similar device. Materials from which the flexible casing 113 may be fabricated include cloth, plastic, rubber, and the like. As above, the pillow 111 can be a resilient flexible material (e.g., foam or rubber).

As shown above, the permeable layer 112 is defined by interconnected strands, filaments, or strips of material interconnected in any one or a combination of manners to define a gauze or a mesh that defines openings that are capable of retaining zeolite particles 114 therein while allowing liquid to flow through. As illustrated, only a few zeolite particles 114 are shown. The permeable layer 112 is attached to the flexible casing 113 along the peripheral edges of the permeable layer. Methods by which the permeable layer 112 may be attached to the flexible casing 113 include, but are not limited to, stitching, gluing, combinations of the foregoing, and the like. Permeable layers 112 may be attached to one surface of the flexible casing 113 or two opposing surfaces of the flexible casing.

To use the pillowcase 110 to treat a bleeding wound, the pillowcase is disposed over a pillow and a victim is placed on the pillowcase 110 such that the bleeding wound contacts the zeolite-filled permeable layer 112. The zeolite particles 114 are either in direct contact with the tissue of the wound or are in direct contact with the blood. The pillow may be strapped to the victim at the wound site using a strapping device such as a belt, an elastic device, hook-and-loop material, combinations of the foregoing devices and materials, and the like.

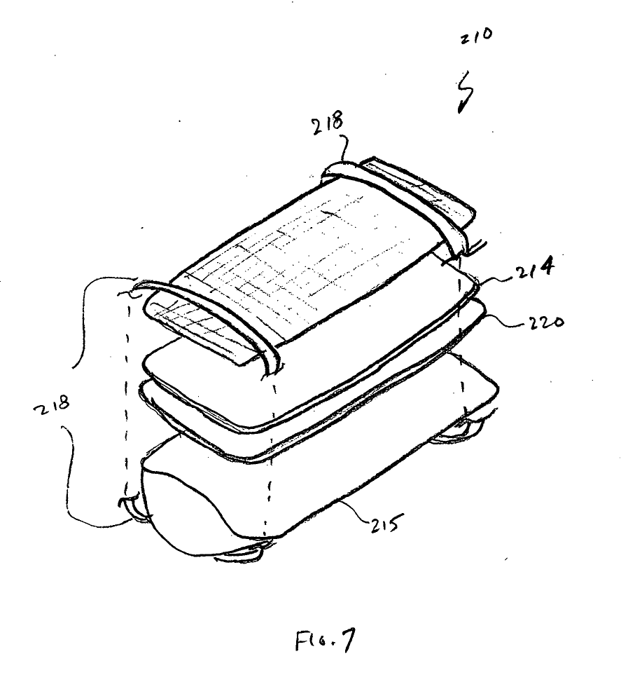

As shown in FIG. 6, a pillow 210 is in the form of a sealed pouch having a first outer surface 212 in the form of a mesh, and an opposed second outer surface 214 in the form of a generally impermeable layer. This embodiment of the present invention can be used as an overlay that rests on top of a conventional pillow, on the mattress of a gurney, or on any surface upon which an injured person is positioned. The first mesh outer surface 212 is positioned so that it comes into contact with a bleeding wound. The sealed pouch 210 can include hook-and-loop fasteners 216 attached to the second outer surface so that it can be relatively immovably positioned on, for example, a conventional pillow 215.

Similarly, as shown in FIG. 7, the sealed pouch 210 can have resilient or elastic straps 218 attached thereto to temporarily secure the sealed pouch to a conventional pillow 215 or the like. In addition, the straps 218 can be used to secure the pouch around that part of a person or animal where a bleeding wounded area is present.

The sealed pouch 210 can also incorporate an absorbent layer 220 located between the second outer surface 214 and the molecular sieve material.

In the preparation of zeolite material (i.e., formation of the material into particle form) for the devices of the present invention, an initial level of hydration of the zeolite may be controlled by the application of heat to the zeolite material either before or after the material is formed into particles. However, it has also surprisingly been found that as the particle size of the zeolite is increased, the moisture content has less of a correlative effect on any exothermia produced as the result of mixing the particlized zeolite in blood. As such, formation of the zeolite material into the zeolite particles may be by extrusion, milling, casting, or the like.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments disclosed in the above detailed description, but that the invention will include all embodiments falling within the scope of the above description.

What is claimed is:

1. An apparatus for promoting the clotting of blood, comprising:
a pillow;
a permeable layer for retaining molecular sieve material in particulate form therein attached to a surface of said pillow, at least a portion of said permeable layer being defined by a mesh having openings therein;
wherein when treating a bleeding wound, contacting said bleeding wound with said permeable layer causes at least a portion of said molecular sieve material to come into contact with blood through said openings.

2. The apparatus of claim 1, wherein said molecular sieve material is a zeolite.

3. The apparatus of claim 2, wherein said zeolite comprises particles having diameters of about 0.2 mm to about 10 mm.

4. The apparatus of claim 1, wherein said mesh is flexible.

6. The apparatus of claim 1, wherein said mesh is conformable to a shape of a wound and can retain said shape of said wound.

7. The apparatus of claim 1, wherein at least one particle of said particulate molecular sieve material protrudes through one of said openings.

8. The apparatus of claim 1, wherein said permeable layer is defined by strands that are interconnected by being woven, intertwined, integrally-formed, or a combination thereof.

9. The apparatus of claim 1, wherein said pillow comprises a resilient core defined by a foam material.

10. A pillow for supporting the head of a person having a head wound, said pillow comprising:
a resilient core; and
a permeable layer for retaining molecular sieve material in particulate form therein attached to at least one surface of said resilient core, at least a portion of said permeable layer being defined by a mesh having openings therein;
wherein when contacting the head wound with said permeable layer, at least a portion of said molecular sieve material comes into contact with blood emanating from the head wound through said openings.

11. The pillow of claim 10, wherein said molecular sieve material is a zeolite.

12. The pillow of claim 10, wherein said resilient core is conformable to a shape of the head of the person having the head wound.

13. The pillow of claim 10, wherein said mesh is conformable to a shape of the head wound and can retain said shape of the head wound.

14. A pillowcase for controlling bleeding of a head wound, comprising:
a flexible casing fittable over a pillow; and
a permeable layer for retaining molecular sieve material in particulate form therein attached to at least one surface of said flexible casing, at least a portion of said permeable layer being defined by a mesh having openings therein;
wherein when contacting the head wound with said permeable layer, at least a portion of said molecular sieve material comes into contact with blood emanating from the head wound through said openings.

15. The pillowcase of claim 14, wherein said molecular sieve material is a zeolite.

16. The pillowcase of claim 14, wherein said flexible casing is closable using a device from the group consisting of zippers, hook-and-loop material, snaps, and combinations of the foregoing.

17. A device for controlling bleeding, said device comprising:
a pouch having a first surface and an opposing second surface; and
a permeable layer for retaining molecular sieve material in particulate form therein attached to at least one of said first surface and said second surface, at least a portion of said permeable layer being defined by a mesh having openings therein;
wherein when treating a bleeding wound, at least a portion of said molecular sieve material comes into contact with blood emanating from said bleeding wound through said openings.

18. The device of claim 17, wherein said molecular sieve material is a zeolite.

19. The device of claim 17, wherein said device is attachable to a pillow.

20. The device of claim 17, wherein said device is an overlay that can be used to support a person at said bleeding wound.

21. A method of treating a bleeding wound, said method comprising the steps of:
providing a pillow having molecular sieve material in particle form retained therein; and
supporting said bleeding wound such that blood flowing from said bleeding wound comes into contact with the molecular sieve material, thereby causing the blood to coagulate.
